(19) United States
(12) Patent Application Publication
(10) Pub. No.: US 2025/0105294 A1
(43) Pub. Date: Mar. 27, 2025

(54) ORGANIC POLYMER CATHODE FOR SECONDARY MAGNESIUM AND SYNTHESIS METHOD THEREOF

(71) Applicant: THE TRUSTEES OF COLUMBIA UNIVERSITY IN THE CITY OF NEW YORK, New York, NY (US)

(72) Inventors: Colin Nuckolls, New York, NY (US); Fay W. Ng, New York, NY (US); Kelsey Harrison, New York, NY (US); Qian Cheng, Beijing (CN); Wenrui Lei, New York, NY (US); Zexin Jin, Zhejiang Province (CN); Haoyu Jiang, New York, NY (US); Qifeng Jiang, New York, NY (US)

(21) Appl. No.: 18/777,255

(22) Filed: Jul. 18, 2024

Related U.S. Application Data
(60) Provisional application No. 63/527,491, filed on Jul. 18, 2023.
(60) Provisional application No. 63/527,499, filed on Jul. 18, 2023.

(51) Int. Cl. H01M 4/60; H01M 4/02; H01M 4/38; H01M 10/0569

(52) U.S. Cl. CPC H01M 4/608; H01M 4/381; H01M 2004/027; H01M 2004/028; H01M 10/0569; H01M 2300/0028

(57) ABSTRACT

A cathode active material having a polymer with helical perylene diimide (hPDI) subunits with the side-chains of the helical perylene diimide (hPDI) subunits removed and a method of manufacturing the cathode active material are provided. A rechargeable battery cell with the polymer as a cathode material, a magnesium metal anode, and an ether-based electrolyte.

### CROSS REFERENCE TO RELATED APPLICATIONS

This application claims priority under 35 U.S.C. § 119(e) to U.S. Provisional Patent Application No. 63/527,491 filed on Jul. 18, 2023 and No. 63/527,499 filed on Jul. 18, 2023, which are incorporated by reference herein in their entirety.

### STATEMENT REGARDING FEDERALLY SPONSORED RESEARCH

This invention was made with government support through the National Science Foundation under Award Number: 2002634, titled “Porous Organic Solid-State Materials for Energy Storage.” The government has certain rights in the invention.

### BACKGROUND

The present disclosure relates to the field of electrochemical cells, including electrolyte materials, electrodes, and other components used in electrochemical cells.

Lithium-ion batteries are the world's predominant energy storage devices and as such have significantly accelerated the development of today's digital information society. Compared to current inorganic electrode materials, organic electrode materials provide unique opportunities to achieve high power density through molecular engineering that can, in principle, yield simultaneously high ionic diffusivity and high specific capacities.

However, one of the major issues regarding organic electrode materials is their tradeoff between specific capacity and cycling stability. Organic molecules, albeit with high capacity, leach into the electrolyte solution during redox reactions, which results in poor cycling stability. Organic polymers can overcome the dissolution issues, but they typically have low charge capacity due to the redox-inactive nature of their linkers and solubilizing side chains.

Beyond these challenges, the low intrinsic electrical conductivity of organic electrode materials (OEM) has thwarted their ability to achieve high rate capability and high areal mass-loading. Consequently, incorporating a high ratio of conductive additives is usually necessary in electrode fabrication for organics to achieve idealized results, which further limits OEMs' technological utility.

There is therefore a need for a polymer material that solves the fundamental issues associated with existing organic electrode materials.

### SUMMARY

In one aspect of the disclosure, a polymer is provided with helical perylene diimide subunits and the side-chains of the helical perylene diimide (hPDI) subunits are removed.

In some embodiments, the polymer has a structure as shown in the following formula:

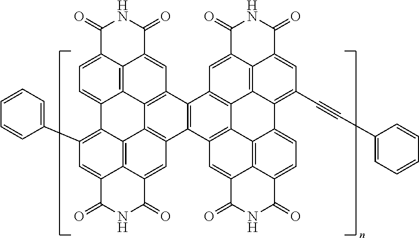

In another embodiment, the polymer has a structure as shown in the following formula:

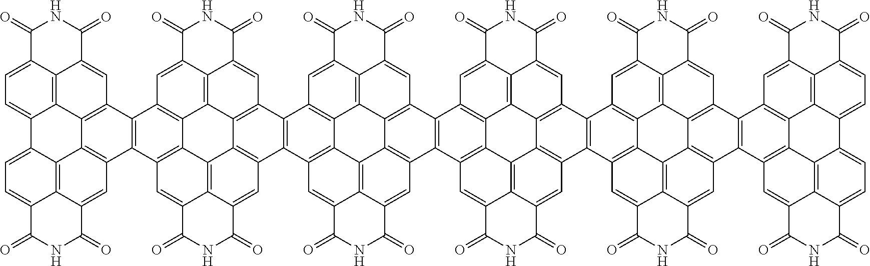

In still another embodiment, the polymer has a structure as shown in the following formula:

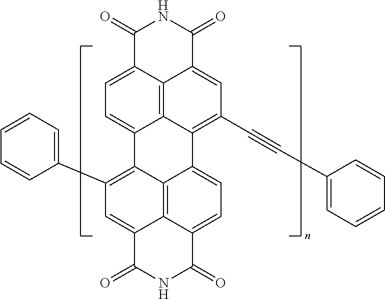

In still another embodiment, the polymer has a structure as shown in the following formula:

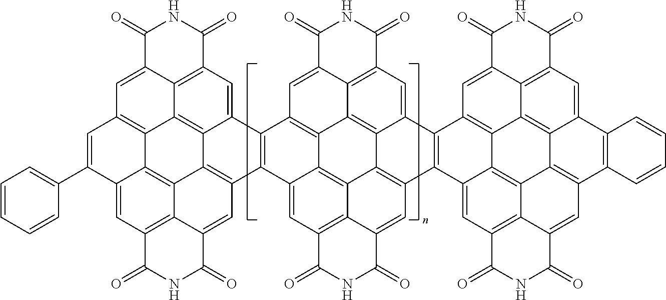

In certain embodiments, the polymer comprises a polymer PTE-alkyne copolymer with an average length between 15 and 200.

One aspect of the disclosure provides for a method of manufacturing the polymer by synthesizing a first intermediate from a perylene-based acid; brominating the first intermediate to form a second intermediate; brominating the second intermediate to form a third intermediate; copolymerizing the second intermediate and the third intermediate to synthesize a perylene-based copolymer; cyclizing the perylene-based copolymer to yield a cyclized polymer; helicizing the cyclized polymer; and removing a side-chain of the helicized polymer.

Yet another aspect of the disclosure provides for a rechargeable battery cell comprising a polymer having the structural formula of:

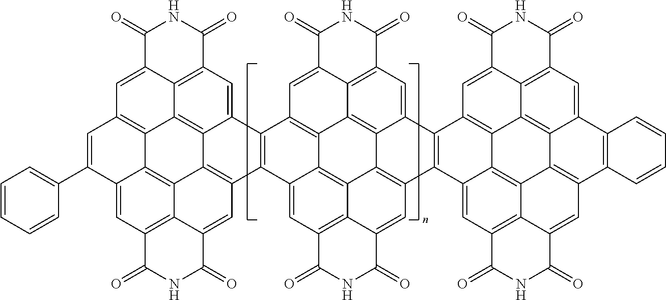

as a cathode material, a magnesium metal anode, and an ether-based electrolyte.

### BRIEF DESCRIPTION OF THE DRAWINGS

Further features and advantages of the present disclosure will become apparent from the following detailed description taken in conjunction with the accompanying figures showing illustrative embodiments of the present disclosure.

FIG. 1 shows the structural formula of a compound comprised of a hPDI[2] monomer according to certain embodiments of the present disclosure.

FIG. 2 shows the structural formula of a compound comprised of a hPDI[6] polymer according to certain embodiments of the present disclosure.

FIG. 3 shows the structural formula of a compound comprised of repeating units of PDI according to certain embodiments of the present disclosure.

FIG. 4 shows the structural formula of a compound comprised of repeating units of hPDI according to certain embodiments of the present disclosure.

FIG. 5A illustrates the synthesis of perylene tetraester (PTE) according to a method of synthesizing an embodiment of the compounds of the present disclosure.

FIG. 5B illustrates the synthesis of bromo perylene tetraester (PTE-Br) according to a method of synthesizing an embodiment of the compounds of the present disclosure.

FIG. 5C illustrates the synthesis of diromo perylene tetraester (PTE-Br₂) according to a method of synthesizing an embodiment of the compounds of the present disclosure.

FIG. 5D illustrates the synthesis of PTE-alkyne copolymer according to a method of synthesizing an embodiment of the compounds of the present disclosure.

FIG. 5E illustrates the synthesis of cyclized hPTE polymer according to a method of synthesizing an embodiment of the compounds of the present disclosure.

FIG. 5F illustrates the synthesis of C5-hPDI polymer according to a method of synthesizing an embodiment of the compounds of the present disclosure.

FIG. 5G illustrates the synthesis of hPDI polymer through the removal of the side-chains of the C5-hPDI polymer according to a method of synthesizing an embodiment of the compounds of the present disclosure.

FIGS. 6A and 6B provide characterization of the coin cells with hPDI[2] monomer as a cathode material. FIG. 6A shows the specific capacity 602 of the coin cell discharged at a 1 C rate over 500 cycles plotted in the lower graph and the coulombic efficiency 601 of the coin cell over the same 500 cycles plotted in the upper graph. FIG. 6B shows the specific capacity 603 of the coin cell and its capacity retention at sequentially higher discharge rates and then back at the initial discharge rate where the coin cell was discharged at a C rate of 1 C for 20 cycles, and a C rate of 2 C, 5 C, 10 C, and 20 C for 10 cycles each and then returned to a C rate of 1 C for another 10 cycles.

FIGS. 7A and 7B provide characterization of the coin cells with hPDI[6] polymer as a cathode material. FIG. 7A shows the specific capacity 702 of the coin cell discharged at a 1 C rate over 500 cycles plotted in the lower graph and the coulombic efficiency 701 of the coin cell over the same 500 cycles plotted in the upper graph. FIG. 7B shows the specific capacity 703 of the coin cell and its capacity retention at sequentially higher discharge rates and then back at the initial discharge rate where the coin cell was discharged at a C rate of 1 C for 20 cycles, and a C rate of 2 C, 5 C, 10 C, and 20 C for 10 cycles each and then returned to a C rate of 1 C for another 10 cycles.

FIGS. 8A and 8B provide characterization of the coin cells with a PDI-alkyne polymer as a cathode material. FIG. 8A shows the specific capacity 802 of the coin cell discharged at a 1 C rate over 500 cycles plotted in the lower graph and the coulombic efficiency 801 of the coin cell over the same 500 cycles plotted in the upper graph. FIG. 8B shows the specific capacity 803 of the coin cell and its capacity retention at sequentially higher discharge rates and then back at the initial discharge rate where the coin cell was discharged at a C rate of 1 C for 20 cycles, and a C rate of 2 C, 5 C, 10 C, and 20 C for 10 cycles each and then returned to a C rate of 1 C for another 10 cycles.

FIGS. 9A and 9B provide characterization of the coin cells with PTE-alkyne copolymers end-capped with a monomer (mono-halide) and a difunctional (bis-halide) as a cathode material. The PTE-alkyne copolymers have average lengths of n=15, n=40, and n=200. FIG. 9A shows the specificity capacity of the PTE-alkyne copolymers hPDI[n=15](902a), hPDI[n=40] (903a), and hPDI[n=200] (904a) plotted against the hPDI[6] polymer (901a) where all four coin cells were discharged at 10 C. FIG. 9B shows the specific capacity of the PTE-alkyne copolymers hPDI[n=15] (902b), hPDI[n=40] (903b), and hPDI[n=200](904b) plotted against the hPDI[6] polymer (901b) where the coin cells were discharged at a C rate of 1 C for 20 cycles, and a C rate of 2 C, 5 C, 10 C, and 20 C for 10 cycles each and then returned to a C rate of 1 C for another 10 cycles.

### DETAILED DESCRIPTION

The present disclosure provides certain compounds that are polymers that contain helical perylene diimide (hPDI) subunits with their side-chains removed. In certain compounds of the present disclosure, the alkynes are not cyclized and are polymers that contain perylene diimide (PDI) subunits.

The present disclosure also provides methods of making the compounds disclosed herein.

The present disclosure also provides cathode materials having the compounds of the present disclosure. Further, the present disclosure also provides for rechargeable battery systems with the cathode materials having the compounds of the present disclosure. Compared to certain conventional technologies, the disclosed subject matter offers a more efficient synthesis process, enabling cost-effective and large-scale production. Notably, the cathode material exhibits exceptional performance in terms of fast rate cycling, allowing for rapid charge and discharge cycles without compromising its overall efficiency, and demonstrates a high-capacity usage efficiency, providing improved utilization of its energy storage capabilities. This contrasts with certain other materials including hPDI[n=3], hPDI[n=4], and hPDI[n=5], and is competitive with hPDI[n=6], a polymer that is limited to small-scale synthesis.

### Compounds

In certain embodiments, the compounds contain helical perylene diimide (hPDI) subunits with their side-chains removed. In certain embodiments, the compound is a polymer.

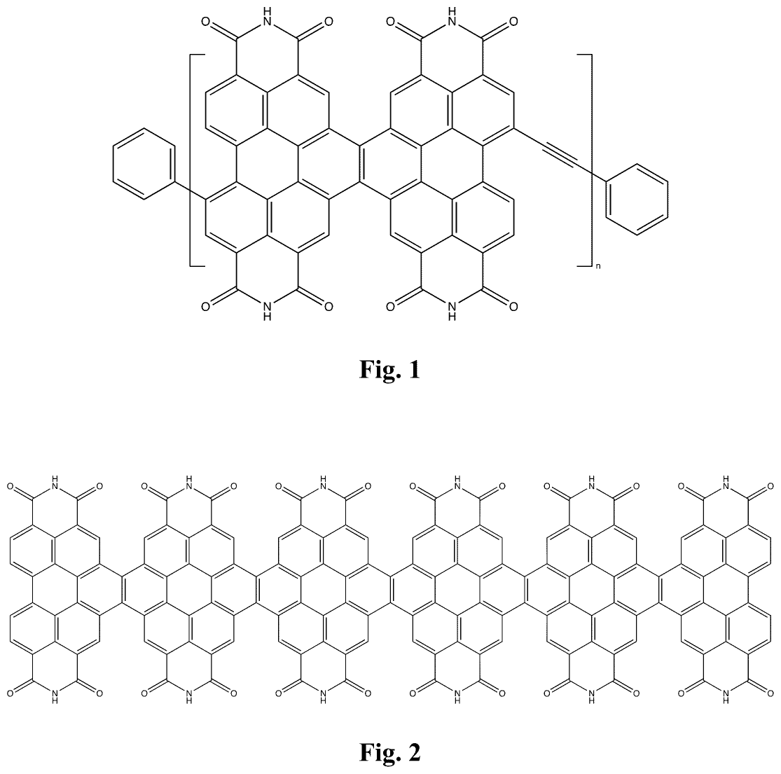

In certain embodiments, the polymer is a hPDI[2] monomer and is ribbon-structure or macromolecular ladder type structure conjugated and contorted at different aromatic sites. Referring now to FIG. 1, in certain embodiments, the polymer has a hPDI[2] monomer and has the structure shown in Formula 1.

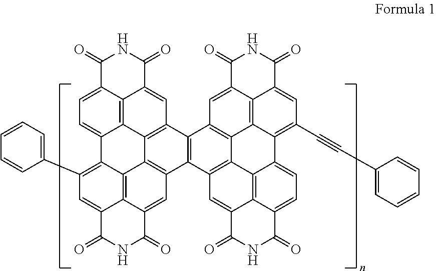

Referring now to FIG. 2, in certain embodiments, the polymer is a hPDI[6] polymer and has the structure shown in Formula 2.

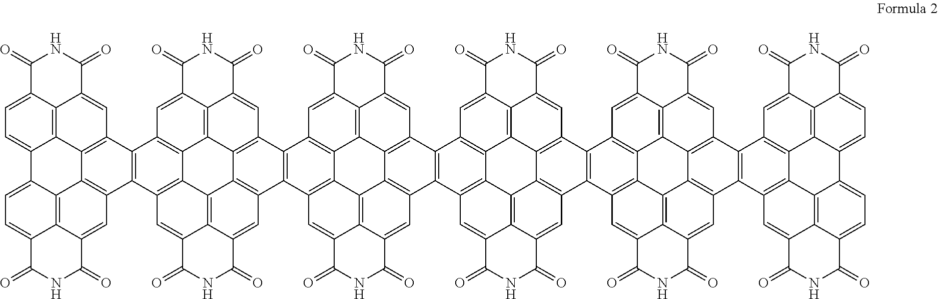

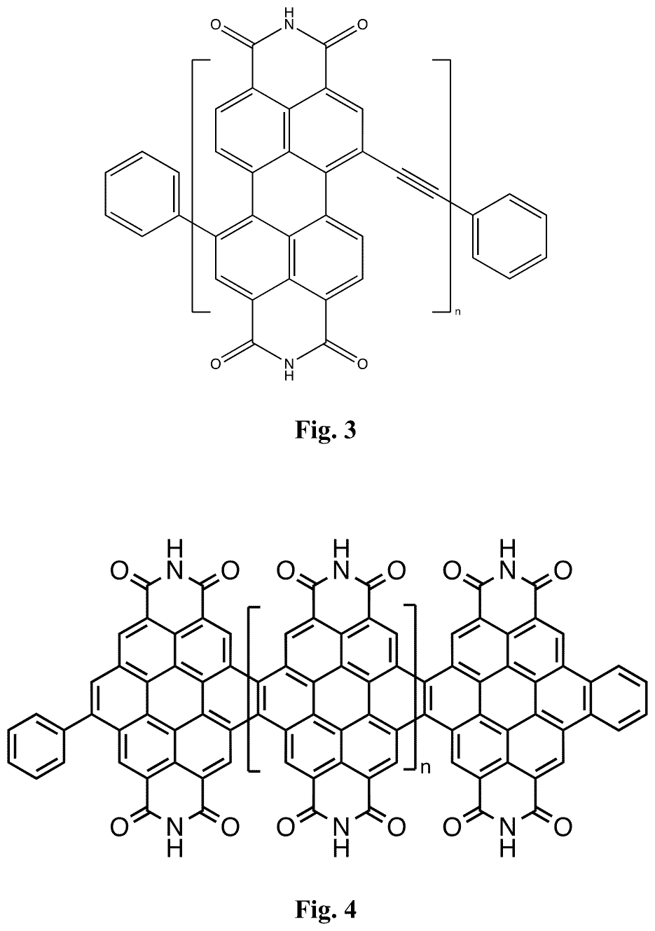

Referring now to FIG. 3, in certain embodiments, the polymer is a PDI-alkyne polymer and has the structure shown in Formula 3.

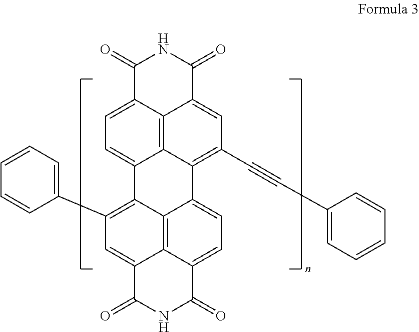

In certain embodiments, the polymer includes repeating units of Formula 3.

Referring now to FIG. 4, in certain embodiments, the polymer includes the structure shown in Formula 4.

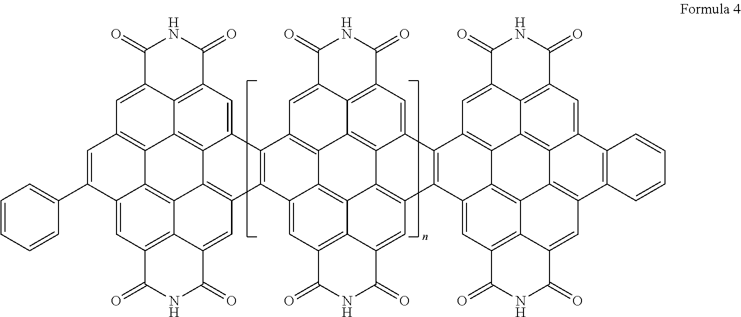

This polymer comprises a PTE-alkyne copolymer, expressed as a [poly(PTE-alkyne).], having an average length of n units. A certain amount of mono functional reagents (e.g., halide or stannane) will result in end groups such as the ones shown in Formula 4. The average length refers to an average molecular weight that is controlled by the ratio of the end-capping monomer (mono-halide) and the difunctional (bis-halide) used in the Stille coupling reaction. In one embodiment, the PTE-alkyne copolymer has 15 units and is expressed as [poly(PTE-alkyne)₁₅] or a hPDI[n=15]. In another embodiment, the PTE-alkyne copolymer has 40 units and is expressed as [poly(PTE-alkyne)₄₀] or hPDI[n=40]. In yet another embodiment, the PTE-alkyne copolymer has 90 units and is expressed as [poly(PTE-alkyne)₉₀] or hPDI[n=90]. In yet another embodiment, the PTE-alkyne copolymer has 200 units and is expressed as [poly(PTE-alkyne)₂₀₀] or hPDI[n=200].

In certain embodiments, the polymers of the present disclosure can be used as cathode materials. The cathode material presents exceptional rate performance at a cycling rate from 0.1 C to 10 C. In certain embodiments, the cathode material has a capacity of up to 97% of its theoretical capacity. In certain embodiments, the cathode material has a capacity of up to 130 mAh/g. In certain embodiments, the cathode material loses less than 13% of its capacity when the current density increases to 10 C.

### Method of Synthesis

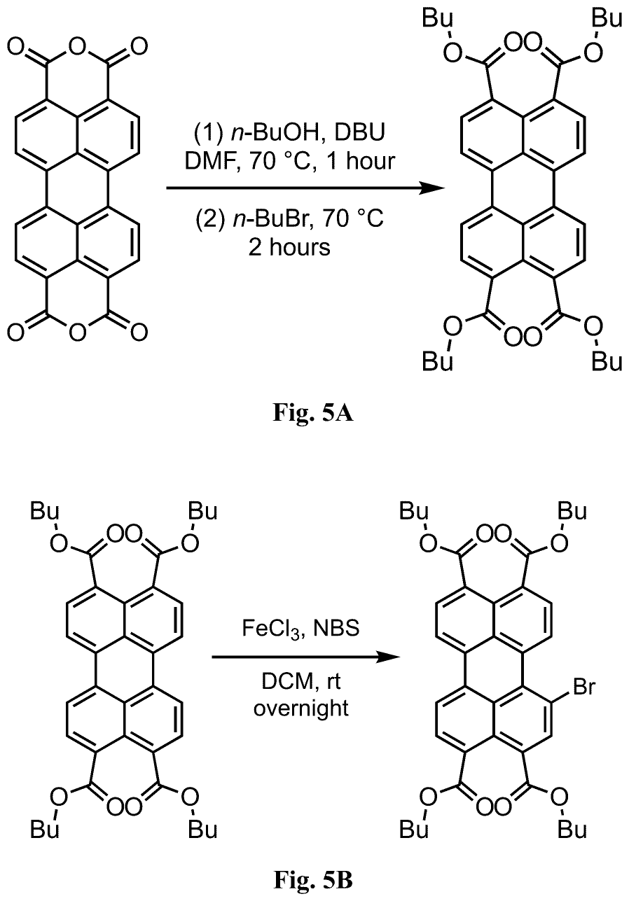

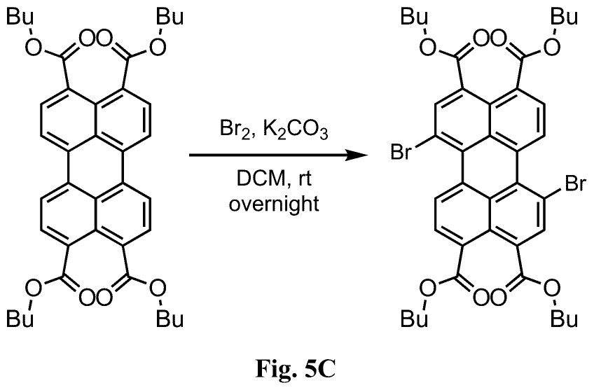

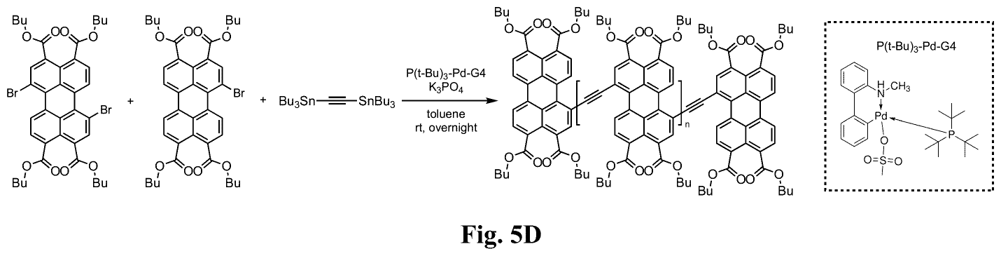

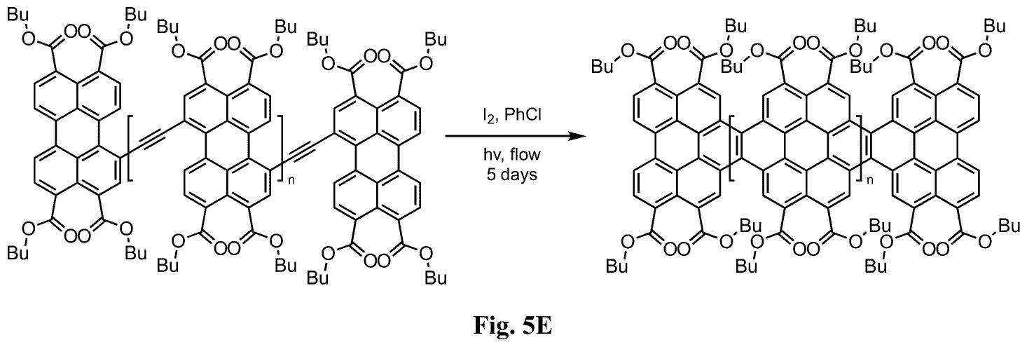

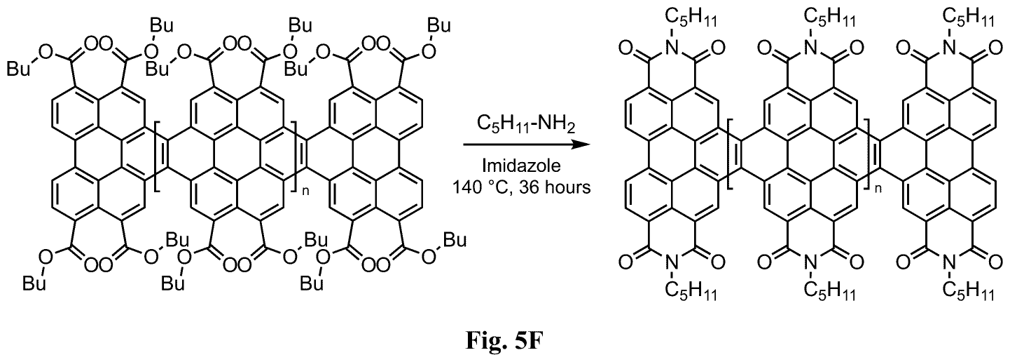

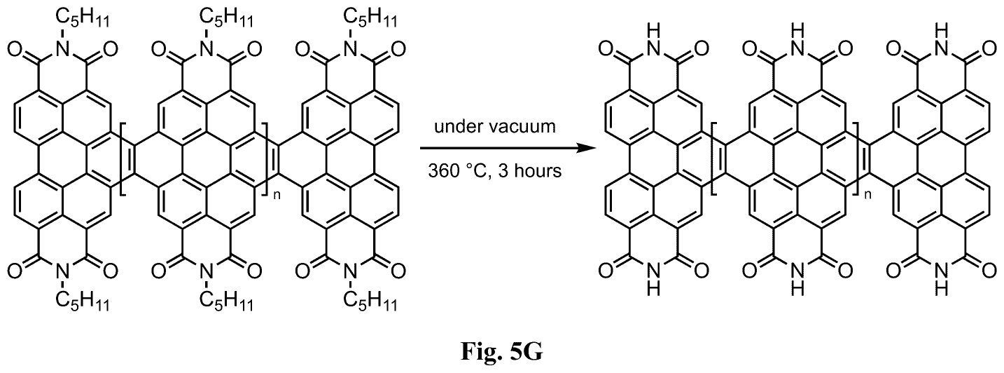

The present disclosure provides a method for synthesizing the presently disclosed compounds. In certain embodiments, the compounds of the present disclosure can be synthesized as illustrated in FIGS. 5A to 5G.

Referring to FIG. 5A, a perylene tetraester (PTE) is synthesized from perylene-3,4,9,10-tetracarboxylic acid dianhydride (PTCDA, 30.0 g, 76.5 mmol, 1.0 eq), 1,8-diazabicyclo[5.4.0]undec-7-ene (45 mL, 306 mmol, 4.0 eq), n-butanol (56 mL, 612 mmol, 8.0 eq), dimethylformamide (400 mL), and 1-bromobutane (66 mL, 612 mmol, 8.0 eq).

Referring to FIG. 5B, from the PTE, a bromo perylene tetraester (PTE-Br) is synthesized. First, the PTE (30 g, 46.0 mmol, 1.0 eq) is mixed with CH₂Cl₂ (390 mL) and FeCl₃ (1.2 g, 7.4 mmol, 0.16 eq) to obtain a solution. The solution is maintained at room temperature overnight. Then, while the solution is vigorously stirred, N-Bromosuccinimide (8.1 g, 45.5 mmol, 0.99 eq) is added. The reaction mixture is quenched with water, extracted with CH₂Cl₂, washed with brine, dried over Na₂SO₄, and concentrated in vacuo. The residue is purified by one-time recrystallization with CH₂Cl₂ and methanol to obtain pure PTE-Br as an orange solid (24.7 g, 33.8 mmol, 73.5%).

Referring to FIG. 5C, a diromo perylene tetraester (PTE-Br₂) is also synthesized from the PTE. The PTE (10.0 g, 15.3 mmol) is dissolved by CH₂Cl₂ (125 mL) in a round bottom flask. K₂CO₃ (15.0 g, 108.5 mmol) and bromine (15 mL) are added to the solution and the reaction mixture is stirred at room temperature overnight. The reaction mixture is then quenched with saturated NaHSO₃ aqueous solution, extracted with CH₂Cl₂, washed with brine, dried over Na₂SO₄, and concentrated in vacuo. The crude is further purified by a silica column with 100% CH₂Cl₂, yielding PTE-Br₂ (9.5 g, 11.7 mmol, 76.5%).

Referring next to FIG. 5D, a PTE-alkyne copolymer is prepared from the PTE-Br (161 mg, 0.25 mmol) and PTE-Br₂ that are synthesized in an earlier step. Under N₂ protection, the PTE-Br₂ (1.0 g, 1.23 mmol), PTE-Br (161 mg, 0.25 mmol), and Bis(trin-butylstannyl) acetylene (745 mg, 1.23 mmol) are dissolved in toluene (50 mL). P(t-Bu)₃ Pd G4 (18 mg, 31 μmol) and K₃PO₄ (157 mg, 1.23 mmol) are added to the solution and the reaction mixture is stirred at room temperature overnight under N₂ protection. The solution is poured into methanol (1 L) and the precipitates are purified by a Soxhlet extraction process where different solvents are used. The chloroform-extracted fraction is collected and dried under vacuum yielding the PTE-alkyne copolymer (709 mg, 85.3%).

Referring then to FIG. 5E, a cyclized hPTE polymer is synthesized from the PTE-alkyne copolymer (709 mg) prepared in an earlier step. The PTE-alkyne copolymer is dissolved in 300 mL chlorobenzene solution of iodine (concentration of I₂ in PhCl: 1.5 mg/mL) and flow-cyclized with LED light for 5 days. The reaction mixture is dried under vacuum, redissolved in minimum volume of chloroform and precipitated from hexane, yielding cyclized hPTE polymer (650 mg, 92%).

Referring next to FIG. 5F, next a C5-hPDI polymer is synthesized. The cyclized hPTE polymer (650 mg) formed in the previous steps is combined with excess imidazole (5 g, used as solvent and base when melted at 140° C.) and excess 3-aminopentane (1.0 g) in a 25 mL vial with stir bar (no need under nitrogen). The reaction mixture is stirred at 140° C. for 36 hours and then the hot mixture is poured into 3N HCl solution. The precipitates are collected via vacuum filtration, washed with water, methanol and acetone, and dried under vacuum to yield the C5-hPDI polymer (505 mg, 95%).

Referring finally to FIG. 5G, in a final step, the side chain of the C5-hPDI polymer is removed to the yield a hPDI polymer. The C5-hPDI polymer (500 mg) is loaded in a one-dram vial and placed into a quartz tube. It is then subjected to heat at 360° C. under vacuum for 3 hours in a tube furnace, yielding the hPDI polymer (366 mg, 98%).

### Rechargeable Cell

The disclosed subject matter further provides a lithium-ion battery system that includes a cathode formulated with the cathode material disclosed above. Certain rechargeable magnesium batteries suffer from issues of irreversibility and slow charging rates limited by strong coulombic interactions with the divalent Mg-ion in electrolytes and cathode materials. In contrast, the disclosed subject matter overcomes these challenges with a novel organic cathode material free of rare or toxic elements with a flexible structure capable of fast and highly reversible Mg-ion diffusion paired with a simple salt electrolyte.

In certain embodiments, the rechargeable battery system includes a magnesium metal anode, an ether-based electrolyte, and a cathode formulated with the cathode materials disclosed above. In certain embodiments, the ether-based electrolyte includes Mg(TFSI)₂/MgCl₂/AlCl₃ electrolyte.

In certain embodiments, the cathode comprises a hPDI material, carbon black, and polyvinylidene difluoride (PVDF) on carbon paper synthesized using methods for synthesizing cathodes well known to a person of ordinary skill in the art.

The ether-based electrolyte can be a magnesium bis(trifluoromethanesulfonyl)imide (Mg(TFSI)₂) based electrolyte, a magnesium dichloride (MgCl₂) based electrolyte, or an aluminum chloride (AlCl₃) based electrolyte. In one embodiment, the ether-based electrolyte comprises 0.5 M Mg(TFSI)₂, 1-methoxy-2-propylamine, and dimethoxyethane (DME).

The description herein illustrates the principles of the disclosed subject matter. Various modifications and alterations to the described embodiments will be apparent to those skilled in the art in view of the teachings herein. Further, it should be noted that the language used herein has been selected for readability rather than to delineate or limit the disclosed subject matter. Accordingly, the disclosure herein is intended to be illustrative, but not limiting, of the scope of the disclosed subject matter. Moreover, the principles of the disclosed subject matter can be implemented in various configurations of hardware and/or software, and are not intended to be limited in any way to the specific embodiments presented herein.

### Examples

The presently disclosed subject matter will be better understood by references to the following Examples. The Examples are provided as merely illustrative of the disclosed methods and systems, and should not be considered as a limitation in any way.

### Example 1—Synthesis of Perylene Tetraester (PTE)

A 1 L round-bottom flask was charged with perylene-3,4,9,10-tetracarboxylic acid dianhydride (PTCDA, 30.0 g, 76.5 mmol, 1.0 eq), 1,8-diazabicyclo[5.4.0]undec-7-ene (45 mL, 306 mmol, 4.0 eq), n-butanol (56 mL, 612 mmol, 8.0 eq), and dimethylformamide (400 mL). The mixture was stirred for 1 hour at 70° C. 1-bromobutane (66 mL, 612 mmol, 8.0 eq) in 200 mL dimethylformamide was added and stirring was continued at 70° C. for 2 hours. The mixture was allowed to cool and then poured into 3 L of water. The resulting slurry was filtered and the solids were washed with water, and then a minimal amount of methanol (approximately 1 L). The orange solid was dried and collected to yield pure PTE as an orange solid (48.1 g, 73.7 mmol, 96%).

### Example 2—Synthesis of Bromo Perylene Tetraester (PTE-Br)

N-Bromosuccinimide (8.1 g, 45.5 mmol, 0.99 eq) was added in five portions to a vigorously stirred solution containing PTE (30 g, 46.0 mmol, 1.0 eq), CH₂Cl₂ (390 mL), and FeCl₃ (1.2 g, 7.4 mmol, 0.16 eq) at room temperature for overnight. The reaction mixture was quenched with water, extracted with CH₂Cl₂, washed with brine, dried over Na₂SO₄, and concentrated in vacuo. The residue was purified by one-time recrystallization with CH₂Cl₂ and methanol to afford pure PTE-Br as an orange solid (24.7 g, 33.8 mmol, 73.5%).

### Example 3—Synthesis of Diromo Perylene Tetraester (PTE-Br₂)

PTE (10.0 g, 15.3 mmol) was dissolved by CH₂Cl₂ (125 mL) in a round bottom flask. K₂CO₃ (15.0 g, 108.5 mmol) and bromine (15 mL) was added to the solution and the reaction mixture was stirred at room temperature overnight. The reaction mixture was quenched with saturated NaHSO₃ aqueous solution, extracted with CH₂Cl₂, washed with brine, dried over Na₂SO₄, and concentrated in vacuo. The crude was further purified by a silica column with 100% CH₂Cl₂ yielding PTE-Br₂ as a bright yellow solid (9.5 g, 11.7 mmol, 76.5%).

### Example 4A—Synthesis of PTE-Alkyne Copolymer [poly(PTE-alkyne)₁₅]

A polymer with 15 units [poly(PTE-alkyne)₁₅] was synthesized. Under N₂ protection, PTE-Br₂ (1.0 g, 1.23 mmol), PTE-Br (161 mg, 0.25 mmol) and Bis(trin-butylstannyl) acetylene (745 mg, 1.23 mmol) were dissolved in toluene (50 mL). P(t-Bu)₃ Pd G4 (18 mg, 31 μmol) and K₃PO₄ (157 mg, 1.23 mmol) were added to the solution and the reaction mixture was stirred at room temperature overnight under N₂ protection. The solution was poured into methanol (1 L) and the precipitates were purified by Soxhlet extraction (methanol for 4 hours, acetone for 16 hours, hexane for 4 hours and chloroform for 4 hours). The chloroform-extracted fraction was collected and dried under vacuum yielding poly(PTE-alkyne)₁₅ as a dark purple solid (709 mg, 85%).

### Example 4B—Synthesis of PTE-Alkyne Copolymer [poly(PTE-alkyne)₄₀]

A polymer with 40 units [poly(PTE-alkyne)₄₀] was synthesized. Under N₂ protection, PTE-Br₂ (1.0 g, 1.23 mmol), PTE-Br (81 mg, 0.12 mmol) and Bis(trin-butylstannyl) acetylene (745 mg, 1.23 mmol) were dissolved in toluene (50 mL). P(t-Bu)₃ Pd G4 (18 mg, 31 μmol) and K₃PO₄ (157 mg, 1.23 mmol) were added to the solution and the reaction mixture was stirred at room temperature overnight under N₂ protection. The solution was poured into methanol (1 L) and the precipitates were purified by Soxhlet extraction (methanol for 4 hours, acetone for 16 hours, hexane for 4 hours and chloroform for 4 hours). The chloroform-extracted fraction was collected and dried under vacuum yielding poly(PTE-alkyne)₄₀ as a dark purple solid (715 mg, 86%).

### Example 4C—Synthesis of PTE-Alkyne Copolymer [poly(PTE-alkyne)₉₀]

A polymer with 90 units [poly(PTE-alkyne)₉₀] was synthesized. Under N₂ protection, PTE-Br₂ (1.0 g, 1.23 mmol), PTE-Br (40 mg, 0.06 mmol) and Bis(trin-butylstannyl) acetylene (745 mg, 1.23 mmol) were dissolved in toluene (50 mL). P(t-Bu)₃ Pd G4 (18 mg, 31 μmol) and K₃PO₄ (157 mg, 1.23 mmol) were added to the solution and the reaction mixture was stirred at room temperature overnight under N₂ protection. The solution was poured into methanol (1 L) and the precipitates were purified by Soxhlet extraction (methanol for 4 hours, acetone for 16 hours, hexane for 4 hours and chloroform for 4 hours). The chloroform-extracted fraction was collected and dried under vacuum yielding poly(PTE-alkyne)₉₀ as a dark purple solid (735 mg, 88%).

### Example 4D—Synthesis of PTE-Alkyne Copolymer [poly(PTE-alkyne)₂₀₀]

A polymer with 200 units [poly(PTE-alkyne)₂₀₀] was synthesized. Under N₂ protection, PTE-Br₂ (1.0 g, 1.23 mmol), PTE-Br (40 mg, 0.06 mmol) and Bis(trin-butylstannyl) acetylene (745 mg, 1.23 mmol) were dissolved in xylene (50 mL). P(t-Bu)₃ Pd G4 (18 mg, 31 μmol) and K₃PO₄ (157 mg, 1.23 mmol) were added to the solution and the reaction mixture was stirred at room temperature overnight under N₂ protection. The solution was poured into methanol (1 L) and the precipitates were purified by Soxhlet extraction (methanol for 4 hours, acetone for 16 hours, hexane for 4 hours and chloroform for 4 hours). The chloroform-extracted fraction was collected and dried under vacuum yielding poly(PTE-alkyne)₂₀₀ as a dark purple solid (756 mg, 91%).

### Example 5—Synthesis of Cyclized hPTE Polymer

PTE-alkyne copolymer (709 mg) was dissolved in 300 mL chlorobenzene solution of iodine (concentration of I₂ in PhCl: 1.5 mg/mL) and flow-cyclized with LED light for 5 days. The reaction mixture was dried under vacuum, redissolved in minimum volume of chloroform and precipitated from hexane, which yielded cyclized hPTE polymer as a red solid (650 mg, 92%).

### Example 6—Synthesis of C5-hPDI Polymer

Cyclized hPTE polymer (650 mg) was combined with excess imidazole (5 g, used as solvent and base when melted at 140° C.) and excess 3-aminopentane (1.0 g) in a 25 mL vial with stir bar (no need under nitrogen). The reaction mixture was stirred at 140° C. for 36 hours and then the hot mixture was poured into 3N HCl solution. The precipitates were collected via vacuum filtration, washed with water, methanol and acetone, and dried under vacuum yielding C5-hPDI polymer as a dark green solid (505 mg, 95%).

### Example 7—Side Chain Removal to Synthesize hPDI Polymer

C5-hPDI polymer (500 mg) was loaded in a one-dram vial and placed into a quartz tube, then subjected to heat at 360° C. under vacuum for 3 hours in a tube furnace, which yielded hPDI polymer as a black solid (366 mg, 98%).

### Example 8—Cathode Fabrication

Each of the polymers synthesized—the hPDI[2] monomer based polymer, the hPDI[6] polymer, the PDI-alkyne polymer, the hPDI[n=15], the hPDI[n=40], and the hPDI[n=200]—were prepared into a cathode. The polymers were ground in an agate mortar and pestle. The material was combined with carbon black and polyvinylidene difluoride (PVDF) in a 70/20/10 mass ratio. Approximately 3.6 times the total mass of the mixture was then added to n-methyl-2-pyrrolidone (NMP) to form a slurry. The slurry was stirred for several hours. Carbon paper (AvCarb MGL190) was cut into rectangles (˜0.5-1 cm by 1-2 cm) and the resulting substrates were sonicated in 1 M H₂SO₄ for 20 minutes to remove any residue or carbon dust. The substrates were then washed with DI water and acetone, dried at 60° C. under vacuum, and weighed on an analytical balance (0.001 mg precision). The slurry was manually deposited on the bottom half of the substrates and pressed using a spatula. The electrode was dried at 70° C. under vacuum overnight. The electrode was then weighed on an analytical balance to obtain the active material mass. Typically, the mass loading was 2-3 mg/cm². Finally, prior to testing, the electrode was soaked in 6 M aqueous KOH solution under static vacuum for several hours.

### Example 9—Anode Fabrication

A 1.8 cm² magnesium metal anode was prepared. From a magnesium metal sheet, a circular sheet having a diameter of 1.51 cm (15.2 mm) was precisely cut out. The circular cut magnesium sheet was checked for uniformity and the edges were smoothed with sandpaper or a metal file to remove burrs or rough spots. Very fine (2000 grit) sandpaper was used to smooth the surface of the magnesium anode.

### Example 10—Electrolyte Preparation

An electrolyte containing 0.5 M Mg(TFSI)₂ in a mixture of 1-methoxy-2-propylamine and DME (dimethoxyethane) was prepared. 50 mL of 1-methoxy-2-propylamine and 50 mL of DME were prepared. To prepare 0.5 M concentration of Mg(TFSI)₂ in 100 ml of solution, 29.22 g of Mg(TFSI)₂ was measured out. In a volumetric flask, the measured 1-methoxy-2-propylamine and DME were added. A magnetic stir bar was then placed in the flask and the mixture was stirred. Mg(TFSI)₂ was then slowly added to the stirring mixture of 1-methoxy-2-propylamine and DME. Stirring was continued until the Mg(TFSI)₂ completely dissolved.

### Example 11—Coin Cell Fabrication

Coin cell batteries each comprising a cathode, a magnesium metal anode, electrolyte, and glass separators were assembled. The hPDI polymer cathodes were fabricated as described above, using carbon paper (AvCarb MGL190) as the current collector. Specifically, cathodes with the hPDI[2] monomer, cathodes with the hPDI[6] polymer, cathodes with the PDI-alkyne polymer, and hPDI[n=15], hPDI[n=40], and hPDI[n=200] polymer cathodes (i.e., cathodes with repeating hPDI subunits) were prepared.

Coin cells with each of the different cathodes were also prepared. The coin cells, of the CR2032 size, were assembled according to established protocols. The coin cells were 2032 coin cells, which have a diameter of 20 mm and a thickness of 3.2 mm. The different hPDI-based cathodes were fabricated as described above. A magnesium metal anode of 1.8 cm², which has a diameter of 15.2 mm (radius of 0.76 mm) that fits the 20 mm diameter of the 2032 coin cell, was also prepared as described above. The cathode and anode were stacked with two glass fiber separators and wet with 0.5 M Mg(TFSI)₂ electrolyte. A steel spacer and spring were then added, and the stack was enclosed in a steel button cell shell and sealed with Parafilm to prevent electrolyte evaporation. The cell was then tested using a standard spring-loaded clip connected to the potentiostat.

### Example 12—Capacity Measurement

The specific capacity, energy, and power of the coin cells were calculated from the GCD data (discharge cycle). The specific capacity is calculated using equation 1

$${Q}_{s} = \frac{i \cdot t}{{m}_{c}} \qquad ( Equation 1 )$$

where i is the current, t is the cycle time, and m_{c} is the mass of active material on the cathode.

To calculate the specific energy and power of the cathode, the total mass of all cell components were used.

Specific energy is calculated using equation 2

$${E}_{s} = \frac{i \cdot t \cdot Δ E}{{m}_{total}} \qquad ( Equation 2 )$$

where i is the current, t is the cycle time, ΔE is the change in potential, and mₜₒₜₐₗ is given by equation 7

mₜₒₜₐₗ=mₐ+m_{c}+mₑ

where mₐ is the mass of the anode and mₑ is the theoretical mass of the electrolyte required to supply ions for the measured discharge Q.

Specific power was calculated using equation 3

$${P}_{s} = \frac{i \cdot Δ E}{{m}_{total}} \qquad ( Equation 3 )$$

The coin cells was then discharged over 500 cycles at 1 C as well as discharged at different C rates for 70 cycles.

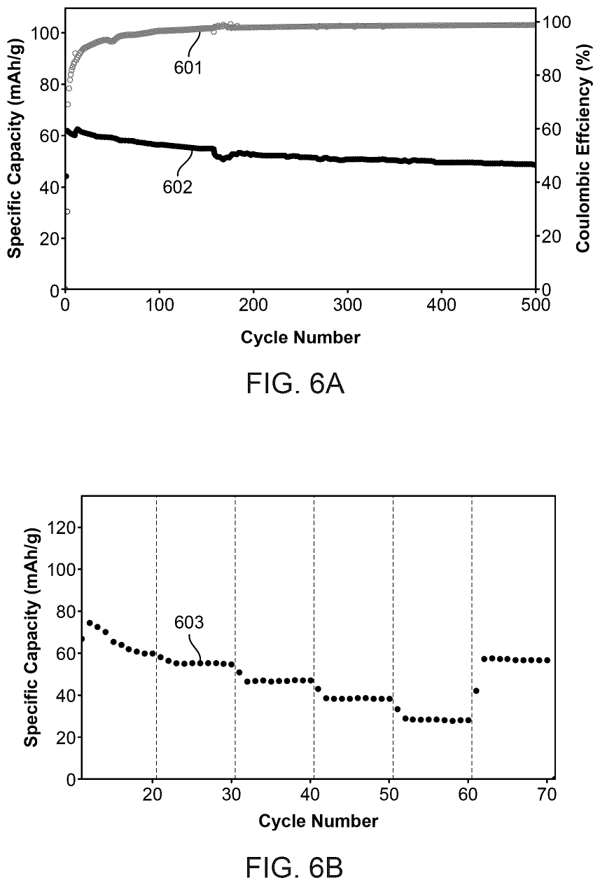

The coin cell was then discharged over 500 cycles at 1 C as well as discharged at different C rates for 70 cycles like the hPDI material in Example 1. Referring now to FIG. 6A, the specific capacity 602 of the coin cell was measured when discharged over 500 cycles and plotted; the coulombic efficiency 601 of the coin cell was also tracked over the 500 cycles. The coin cell with the hPDI[2] monomer as a cathode material exhibited an initial discharge capacity of 64 mAh/g. The specific capacity decreases to 51 mAh/g after 500 cycles at a 1 C discharge rate. Referring now to FIG. 6B, the specific capacity 603 of the coin cell was measured when discharged over 70 cycles at a C rate of 1 C for 20 cycles, and a C rate of 2 C, 5 C, 10 C, 20 C, and 1 C for 10 cycles each. When discharged at the high rate of 20 C between cycles 51 and 60, the coin cell exhibited a specific capacity of 30 mAh/g. And when the coin cell was switched back to a discharge rate of 1 C in cycles 61 to 70, the coin cell had a specific capacity of 60 mAh/g, close to the specific capacity measured in cycles 1 through 20 when first discharged at a 1 C rate.

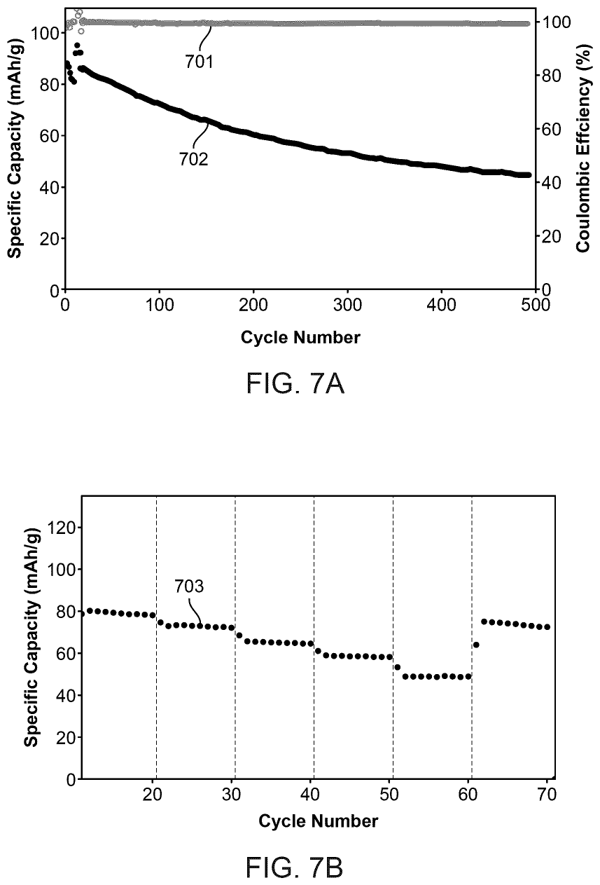

Referring now to FIG. 7A, the specific capacity 702 of the coin cell was measured when discharged over 500 cycles and plotted; the coulombic efficiency 701 of the coin cell was also tracked over the 500 cycles. The coin cell with the hPDI[6] polymer as a cathode material exhibited an initial discharge capacity of 88 mAh/g. The specific capacity decreases to 42 mAh/g after 500 cycles at a 1 C discharge rate. Referring now to FIG. 7B, the specific capacity 703 of the coin cell was measured when discharged over 70 cycles at a C rate of 1 C for 20 cycles, and a C rate of 2 C, 5 C, 1° C., 20 C, and 1 C for 10 cycles each. When discharged at the high rate of 20 C between cycles 51 and 60, the coin cell exhibited a specific capacity of 50 mAh/g. And when the coin cell was switched back to a discharge rate of 1 C in cycles 61 to 70, the coin cell had a specific capacity of 80 mAh/g, close to the specific capacity measured in cycles 1 through 20 when first discharged at a 1 C rate.

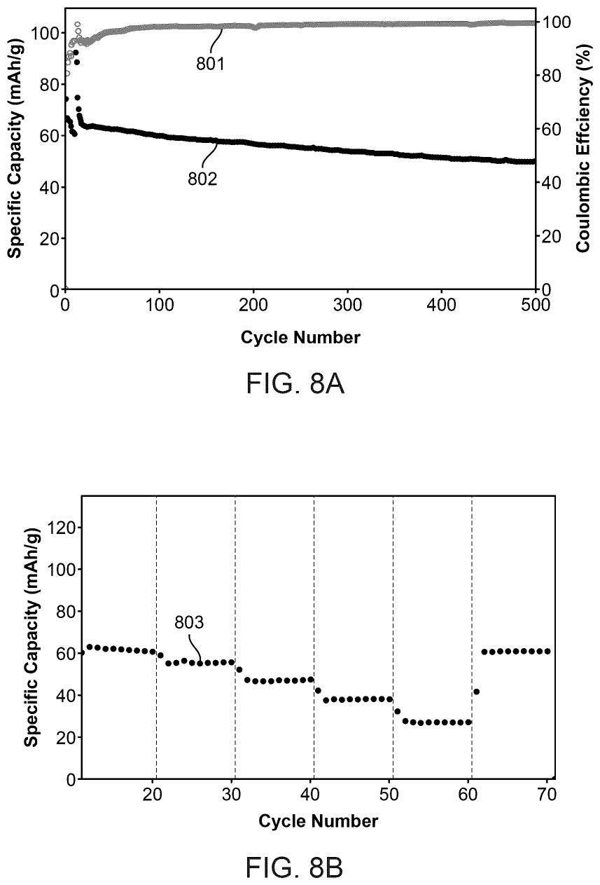

The coin cell was then discharged over 500 cycles at 1 C as well as discharged at different C rates for 70 cycles like the hPDI material in Example 1. Referring now to FIG. 8A, the specific capacity 802 of the coin cell was measured when discharged over 500 cycles and plotted; the coulombic efficiency 801 of the coin cell was also tracked over the 500 cycles. The coin cell with the PDI-alkyne polymer having the structural formula in FIG. 3 as a cathode material exhibited an initial discharge capacity of 68 mAh/g. The specific capacity decreased to 50 mAh/g after 500 cycles at a 1 C discharge rate. Referring now to FIG. 8B, the specific capacity 803 of the coin cell was measured when discharged over 70 cycles at a C rate of 1 C for 20 cycles, and a C rate of 2 C, 5 C, 10 C, 20 C, and 1 C for 10 cycles each. When discharged at the high rate of 20 C between cycles 51 and 60, the coin cell exhibited a specific capacity of 30 mAh/g. And when the coin cell was switched back to a discharge rate of 1 C in cycles 61 to 70, the coin had a specific capacity of 60 mAh/g, close to the specific capacity measured in cycles 1 through 20 when first discharged at a 1 C rate.

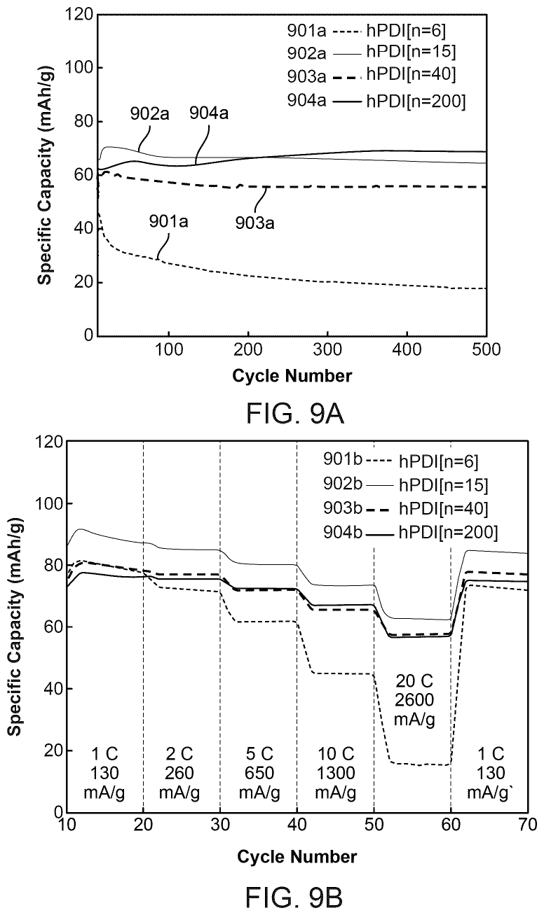

Referring now to FIG. 9A, the specific capacity of the coin cells with hPDI[6] (901a), hPDI[n=15] (902a), hPDI[n=40] (903a), and hPDI[n=200] (904a) as the cathode material were measured when discharged at a 10 C rate over 500 cycles and plotted. The hPDI[n=15] (902a) exhibited a specific capacity of as high as 70 mAh/g without degrading significantly over 500 cycles. The hPDI[n=40] (903a) exhibited a specific capacity of as high as 64 mAh/g without degrading significantly over 500 cycles. The hPDI[n=200] (904a) exhibited a specific capacity of as high as 70 mAh/g without degrading significantly over 500 cycles. Referring now to FIG. 9B, the specific capacity of the coin cells were measured when discharged over 70 cycles at a C rate of 1 C for 20 cycles, and a C rate of 2 C, 5 C, 10 C, 20 C, and 1 C for 10 cycles each. When discharged at the high rate of 20 C between cycles 51 and 60, the coin cells for hPDI[n=15] (902b), hPDI[n=40](903b), and hPDI[n=200] (904b) exhibited a specific capacity of 61 mAh/g, 57 mAh/g, and 57 mAh/g respectively. And when the coins cell were switched back to a discharge rate of 1 C in cycles 61 to 70, the coin cells for hPDI[n=15] (902b), hPDI[n=40] (903b), and hPDI[n=200] (904b) had a specific capacity of 87 mAh/g, 80 mAh/g, and 78 mAh/g respectively, close to the specific capacity measured in cycles 1 through 20 when first discharged at a 1 C rate.

What is claimed is:

1. A polymer, comprising:
helical perylene diimide (hPDI) subunits,
wherein side-chains of the helical perylene diimide (hPDI) subunits are removed.

2. The polymer of claim 1, wherein the polymer comprises monomers of two of the helical perylene diimide (hPDI) subunits.

3. The polymer of claim 1, wherein the polymer has a structure as shown in this formula:

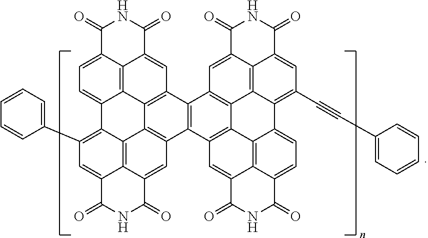

4. The polymer of claim 1, wherein the polymer comprises six helical perylene diimide (hPDI) subunits.

5. The polymer of claim 1, wherein the polymer has a structure as shown in this formula:

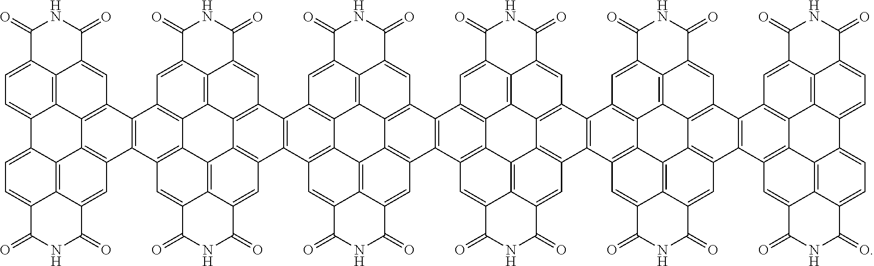

6. The polymer of claim 1, wherein the polymer comprises at least 15 but less than 200 of the helical perylene diimide (hPDI) subunits on average.

7. The polymer of claim 1, wherein the polymer has a structure as shown in this formula:

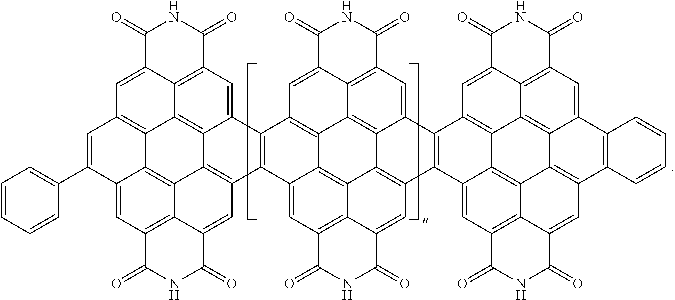

8. A method of forming the polymer of claim 1, comprising:
synthesizing a first intermediate from a perylene-based acid
brominating the first intermediate to form a second intermediate;
brominating the second intermediate to form a third intermediate;
copolymerizing the second intermediate and the third intermediate to synthesize a perylene-based copolymer;
cyclizing the perylene-based copolymer to yield a cyclized polymer;
helicizing the cyclized polymer; and
removing a side-chain of the helicized polymer.

9. A rechargeable battery cell comprising:
the polymer of claim 7 as a cathode material;
a magnesium metal anode; and
an ether-based electrolyte.
